(19) United States
(12) Patent Application Publication
(10) Pub. No.: US 2023/0065999 A1
(43) Pub. Date: Mar. 2, 2023

(54) METHOD AND SYSTEM FOR AUGMENTING CLINICAL NOTES

(71) Applicant: KONINKLIJKEPHILIPS N.V., EINDHOVEN (NL)

(72) Inventors: Nathalie Dorothee Pieternel Leurs, Eindhoven (NL); Daniel Robert Jozef Bolio, Eindhoven (NL); Vincentius Paulus Bull, Eindhoven (NL); Carlijn Andrea Vemooij, Eindhoven (NL); Linda de Goederen, Eindhoven (NL)

(21) Appl. No.: 17/895,117

(22) Filed: Aug. 25, 2022

Related U.S. Application Data
(60) Provisional application No. 63/236,720, filed on Aug. 25, 2021.

(51) Int. Cl. G16H 10/60; G06N 20/00

(52) U.S. Cl. CPC G16H 10/60; G06N 20/00

(57) ABSTRACT

Provided are concepts for generating an augmented clinical note by determining target subject information based on an identified data type of the clinical note, and inserting an identifier to a location of the target information in the clinical note. In particular, a natural language processing algorithm is used to process content of the clinical note in order to identify a data type, which is used to determine target subject information. Using this concept, clinical notes with automatically generated identifiers to locations of target subject information may be obtained. This may obviate the need for manual duplication of information, ultimately reducing errors and a time taken for a user to produce clinical notes, as well as making target subject information easier for a reader to obtain.

### FIELD OF THE INVENTION

The invention relates to the field of generating augmented clinical notes.

### BACKGROUND OF THE INVENTION

In order to share and keep track of patient progress, clinicians add notes and reports to a hospital's electronic medical records (EMR) containing their objective and subjective findings. Typically, these notes are short messages of 2-3 lines that may contain referenced patient information such as vital sign measurements, lab results and images. The information in the notes may be textual copied (“her temperature during the morning round was 38° C.”) or summarized (“the X-Ray image of the left lower arm showed no fracture”).

It is often the case that user mistakes lead to clinical notes containing incorrect or incomplete information. This issue is further compounded by the fact that these notes are often written in a stressful and time-pressured environment. Further, the clinician reading the note may misinterpret the limited textual and numerical information captured in the note.

Moreover, the clinician that writes the note often has to duplicate or summarize patient information in the note (e.g. reference a specific piece of information stored elsewhere in the EMR). Duplication of information is prone to errors, and creating a summary can lead to an incorrect or incomplete summary of the information. In addition, both lead to additional work for the clinician when writing the note.

Indeed, even when errors are not present, it has been shown that clinicians often struggle to access the pertinent information in a timely manner, e.g. due to the information being distributed over multiple systems. Also, reading the notes may take up a significant amount of valuable time, because textual information may be lengthy.

### SUMMARY OF THE INVENTION

The invention is defined by the claims.

According to an aspect of the invention, there is provided a method for generating an augmented clinical note, the method comprising processing, with a natural language processing algorithm, content of a clinical note corresponding to a subject in order to identify a data type; determining target subject information based on the identified data type; and inserting an identifier of a location of the target subject information into the clinical note in order to generate an augmented clinical note.

Proposed embodiments may provide a method of generating augmented clinical notes, such that relevant subject information may be included. By utilising a natural language processing algorithm to analyse the clinical note, target subject information that is relevant to the clinical note may be identified. An identifier of a location of the target subject information may then be included in the note, meaning that manual duplication of the information is not necessary when generating (e.g. writing or dictating) the note. Also, when reading a clinical note, it may not be necessary to switch between various platforms and applications to obtain the target subject information.

It is proposed that an identifier of a location of target subject information is provided in the clinical note. In particular, the content of the clinical note is processed in order to identify a data type. The target subject information is subsequently determined based on the data type. For example, if the identified data type is found to be blood pressure readings, target subject information may relate to recent blood pressure readings. In this case, the user writing/dictating the clinical note may then be referred to the recent BP readings and may then select (part of) the data he/she is referring to. Also, an identifier of a location of blood pressure (BP) readings may be automatically inserted into the clinical note. A user reading the clinical note may use the identifier in order to quickly and easily obtain the BP readings referred to. As a result, writing and reading of clinical notes may be simplified, while also reducing the rate of errors.

Due to the insertion of an address to target (relevant) subject information, the clinical note may be modified so as to provide useful information to a reader. This may improve the speed of access to information when reading the clinical note, which is particularly advantageous in a clinical environment. The automatic insertion of the identifier of target subject information location may also increase the speed of writing clinical notes, by obviating the need for duplication of the target subject information by the user.

By leveraging a natural language processing algorithm, it may be possible to automatically identify a (relevant) data type associated with the clinical note. Accordingly, (relevant) target subject information can be identified, and an identifier of a location of the identified information may be added to the clinical note to generate an augmented clinical note. By inserting an identifier of an address, rather than the target subject information itself, a concise clinical note may still be generated while also ensuring all information is quickly and easily available.

In some embodiments, the method may further comprise a preceding step of determining content of the clinical note as being updated. In this case, the steps of processing the content of the clinical note and determining target subject information are performed responsive to determining that the content has been updated.

By determining when the content of the clinical note has been updated, target subject information can be identified whenever a user adds, removes or modifies content of the clinical note. As a result, target subject information which is relevant to the current state of the content of the clinical note may be identified. Indeed, by identifying a data type and corresponding target subject information in response to the clinical note being updated, it may be ensured that the identifier to an address of target subject information is appropriate for the given clinical note content.

As a user writes or dictates a clinical note, an address of a location of target subject information may be continuously updated. In this way, the inserted identifier is directed towards the most pertinent information relating to the subject.

In some embodiments, the natural language processing algorithm comprises a machine learning algorithm that is trained using a training algorithm configured to receive an array of training inputs and known outputs, wherein the training inputs comprise clinical notes and the known outputs comprise data types.

The natural language processing algorithm may be a machine learning algorithm trained on known data. This leads to an accurate algorithm which is capable of identifying a correct data type of unseen clinical notes. As the identification of a data type is key for determining (relevant) target subject information, the accuracy of the natural language processing algorithm is important.

In some embodiments, the inserted identifier is configured to be user selectable, and the method further comprises communicating the target subject information to a user responsive to the user selecting the identifier.

With the inserted identifier being interactive, it may be possible to convey the target subject information quickly and simply in a readable manner. Advantageously, the reader would not have to reference other systems, application and/or platforms, as the target subject information is provided upon selecting the identifier. Therefore, target subject information may be obtained by the reader in a quick and simple manner, while avoiding potential errors caused by misreading the identifier. In some example embodiments, the interactive identifier may be a hyperlink.

In some embodiments, the method further comprises analyzing content of the clinical note using the natural language processing algorithm to identify a data characterization, and wherein determining target subject information is further based on the data characterization.

A character of data mentioned in the clinical note (for example, a relevant range of desirable data) may be used to obtain more relevant and accurate target subject information. In this way, a more useful augmented clinical note may be obtained.

Further, it may prove advantageous to analyze the clinical note to identify a data characterization. In this way, more relevant target subject information may be determined for insertion into the note via an identifier of an address of the information. For example, a data characterization may be a time stamp of data. In this case, if the user writing the clinical note writes “her temperature in the morning was”, the target subject information may be temperature readings from the morning, rather than all temperature readings which may be obtained if only a data type is used for the determination of target subject information.

In some embodiments, the data characterization comprises one or more of a date stamp, a time stamp, a time window, a variation characteristic, a trend characteristic, a measurement location, a measurement device, a body position of subject, a data storage location, and a measurement characteristic.

Hence, by identifying a data characterization of the clinical note including one of the above metrics, more relevant and accurate target subject information may be identified.

In some embodiments, determining the target subject information is further based on subject context information the subject context information comprising, for example, one or more of a subject history, a subject measurement, a subject condition, a subject treatment plan, a subject location, a subject alarm setting, a subject alarm history, an alarm trigger information, and an outcome of a clinical decision support algorithm (e.g. subject early warning score, hemodynamic stability index, renal failure index or some other indicator of patient status).

Information relevant to the situation the subject is in may be used to obtain more relevant and accurate target subject information. In this way, a more useful augmented clinical note may be obtained.

In some embodiments, the method further comprises obtaining subject context information from at least one of: an electronic medical record (EMR); a picture archiving and communication system (PACS); a lab test; and a medical database.

Information from many sources may be utilized to accurately and completely understand the context of the subject for use in identifying target subject information. Ultimately, this may lead to a more useful augmented clinical note, with an identifier of an address to relevant target subject data.

In some embodiments, determining the target subject information is further based on clinical note context information. A context in which the clinical note is written may provide information useful for determining target subject information which may be relevant to the clinical note.

In some embodiments, the clinical note context information comprises one or more of a clinical note timestamp, an author, an author role, and a location of generating/creating the clinical note.

In some embodiments, inserting the identifier to the location of the target subject information comprises: communicating the target subject information to a user; receiving a confirmation or a rejection corresponding to the target subject information from the user; and inserting the identifier of the location of the target subject information into the clinical note in order to generate the augmented clinical note, responsive to receiving confirmation from the user.

This may enable the user (writer) to have a final say on whether the identifier should be inserted. It may be the case that the determined target subject information is inappropriate or unnecessary, and therefore allowing the user to dictate the final insertion may prove particularly advantageous. This may also aid the user in understanding what they have written, and to review whether what they have written is accurate and what was intended, ultimately reducing errors.

In some embodiments, the method may further comprise: receiving modified target subject information from the user responsive to communicating the relevant subject information to the user; and inserting an identifier of a location of the modified target subject information into the clinical note in order to generate the augmented clinical note.

It may be the case that the recommended target subject information is not precisely what the user wishes to be referenced in the clinical note. Therefore, by enabling the user to modify the target subject information may lead to a more useful augmented clinical note. Indeed, it may be the case that the method allows the user to choose between a selection of the most relevant target subject information. Overall, this improves the accuracy and suitability of the augmented clinical note.

According to another aspect of the invention, there is provided a computer program comprising computer program code means which is adapted, when said computer program is run on a computer, to implement the method for generating an augmented clinical note.

According to another aspect of the invention, there is provided a system for generating an augmented clinical note, the system comprising: an analysis component configured to process, with a natural language processing algorithm, content of a clinical note corresponding to a subject in order to identify a data type; a selection component configured to determine target subject information based on the identified data type; and a modification component configured to insert an identifier of a location of the target subject information into the clinical note in order to generate an augmented clinical note.

In some embodiments, the system further comprises an interface unit configured to determine content of the clinical note as being updated, the analysis component is further configured to analyze the content of the clinical note, and the selection component is further configured to determine target subject information, responsive to the interface unit determining that the content has been updated.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 depicts a block diagram of data flow through a system for generating an augmented clinical note;

FIG. 2 is a flow diagram of a method for generating an augmented clinical note according to an exemplary embodiment;

FIG. 3 is a flow diagram of a method for generating an augmented clinical note according to another exemplary embodiment;

FIG. 4 is a flow diagram of a method for generating an augmented clinical note according to a further exemplary embodiment;

FIG. 5 is a flow diagram of a method for inserting an identifier into a clinical note according to an aspect of an exemplary embodiment;

FIGS. 6A and 6B each depict a user selection screen for inserting an identifier into a clinical note;

FIG. 7 depicts uses of an identifier inserted into a clinical note;

FIG. 8 depicts a use of an identifier inserted into subject information; and

FIG. 9 is a block diagram of a system for generating an augmented clinical note according to an exemplary embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. If the term “adapted to” is used in the claims or description, it is noted the term “adapted to” is intended to be equivalent to the term “configured to”.

It should be understood that the Figures are merely schematic and are not drawn to scale. It should also be understood that the same reference numerals are used throughout the Figures to indicate the same or similar parts.

Implementations in accordance with the present disclosure relate to various techniques, methods, schemes and/or solutions pertaining to generating an augmented clinical note by inserting an identifier of a location of target subject information into a clinical note. According to proposed concepts, a number of possible solutions may be implemented separately or jointly. That is, although these possible solutions may be described below separately, two or more of these possible solutions may be implemented in one combination or another.

Embodiments of the invention aim to provide concepts for generating an augmented clinical note by inserting to an identifier of a location of the target subject information. In particular, target subject information is determined based on an identified data type of the clinical note. The data type is identified by processing content of the clinical note using a natural language processing algorithm. In this way, target subject information which is relevant to the content of the clinical note may be referenced within the note.

In other words, target (relevant) subject information may be added to a clinical note, providing a reader with quick and easy access to related information. Indeed, this may obviate the need for the creator of the clinical note to duplicate information. Ultimately, the inventive concept may aid the creation (e.g. writing and/or dictation) and reading of clinical notes, such that errors are reduced and time is saved.

Ensuring that the author and/or reader of a clinical note has access to all of the accurate and relevant information is key for positive clinical outcomes. The invention provides a solution to the problem of incomplete and inaccurate clinical notes by providing a reference to target subject information in the form of an identifier of a location of the information. Accordingly, time may be saved both during the creating and reading process, and errors may be suppressed.

By utilizing a natural language processing algorithm to process content of the clinical note, the clinical note may be accurately and automatically analyzed. This means that target subject information that is appropriate and relevant to the subject associated with the clinical note may be identified.

By way of explanation, the user/clinician that creates (e.g. writes or dictates) a clinical note often has to duplicate or summarize subject information within the clinical note. Duplication of information is prone to errors, and creating a summary can lead to an incorrect or incomplete summary of the information. In addition, summarization and duplication both lead to additional effort for the clinician when creating the note.

Moreover, the clinician that reads a clinical note often has to learn or refer to the referenced information in order to understand the clinical note. This information may be in the same view as the note, but can also be in a different view, application or system (such as in a PACS or lab test system). In looking up the referenced information, it may be necessary to query the correct subject information, and swap between different views and applications. This is typically inconvenient, time consuming and may even introduce errors.

It has been realized that, by automatically inserting an identifier of a location of the target subject information within the clinical note, the above problems may be overcome. The identifier may be in the form of, for example, a hyperlink. In this way, the identifier may be interactive, and provide the target subject information to the user upon interaction by the user. In some embodiments, the target subject information may be provided in a popup window or in a separate page when the identifier is selected.

Indeed, as clinicians usually work in a time sensitive environment, they typically have little time available to create notes. Therefore, it may be important for the insertion of the identifier to be straightforward. In order to facilitate this, it is beneficial to automatically propose identifiers of locations to (combinations of) target subject information relevant to the content of the clinical note. As such, embodiments may provide proposed identifiers while typing or dictating the note, in a similar way as predictive text works on a smartphone. By way of example, when a note on patient Miller is created and the clinician states “her blood pressure”, a list with relevant blood pressure readings from the patient may be presented. Further, the clinician may then select a (range of) measurement(s) most appropriate for the clinical note.

In other words, while a clinical note is being typed or dictated by a user, the invention may automatically propose identifier of a location of the target subject information. In some embodiments, the target subject information proposed by be based on the following inputs:
(i) Applying a natural language processing algorithm on content of the clinical note to extract a data type. For example, the data type may be a blood pressure, an echocardiogram strip, a lab result, or an ultrasound image.
(ii) Applying a natural language processing algorithm on content of the clinical note to extract a data characterization (of the data type). This may focus on keywords in the note, such as time/date (this morning, yesterday), or a measurement characteristic (high, low, average, trend, variable, unstable, single measurement, multiple measurements, a range of measurements).
(iii) Applying a natural language processing algorithm on content of the clinical note to extract keywords, which may then be used to query a subject health context. For example, this may include a subject history, subject measurements, a list of conditions the subject suffers from, a treatment history of the subject, a subject treatment plan, and subject early warning scores. The information may be stored in electronic medical records (EMR), or in other systems such as PACS, lab test systems and subject monitoring systems.
(iv) Information regarding the context of the clinical note. For example this may include a time/date of the clinical note, an author of the clinical note, a relation/role/responsibility of the author to the subject, and a location of where the clinical note is written.

The above may also be otherwise acquired. For example, a user may enter the information, or it may be determined by external sensors or from external memory.

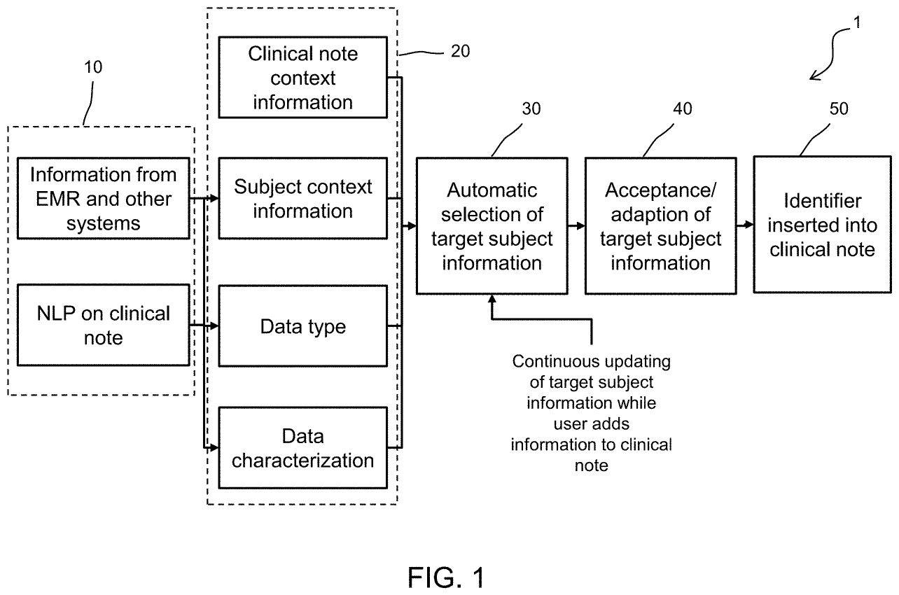

Turning to FIG. 1, there is depicted a block diagram 1 of data flow through a system for generating an augmented clinical note according to an exemplary embodiment. Specifically, the block diagram shows the data flow 1 in order to insert an identifier into the clinical note.

Firstly, raw data 10 is input to the system. This includes the results of processing content of the textual clinical note using natural language processing. This results in a data type of the clinical note, and may also include a data characterization. The natural language processing algorithm may also extract keywords, which may be used to query and combine relevant subject context information.

The raw input data 10 is then processed in order to obtain processed input data 20. The system retrieves the relevant input data including data type and data characterization for the automatic selection of target subject information. Based on keywords extracted from the clinical note in the previous step, subject context information may be retrieved from (external) systems.

The data type, data characterization, subject context information and clinical note context information may then be used to intelligently (for example, by using artificial intelligence) determine the target subject information 30. In some embodiments, this may be a continuous process. In other words, while content is added to the clinical note, the target subject information may be re-evaluated (and updated, if necessary).

The target subject information may then be communicated to the user. The user may accept, reject or manually adapt the target subject information 40. Accordingly, upon acceptance or adaptation, the identifier may then be inserted into the clinical note 50, resulting in an augmented clinical note.

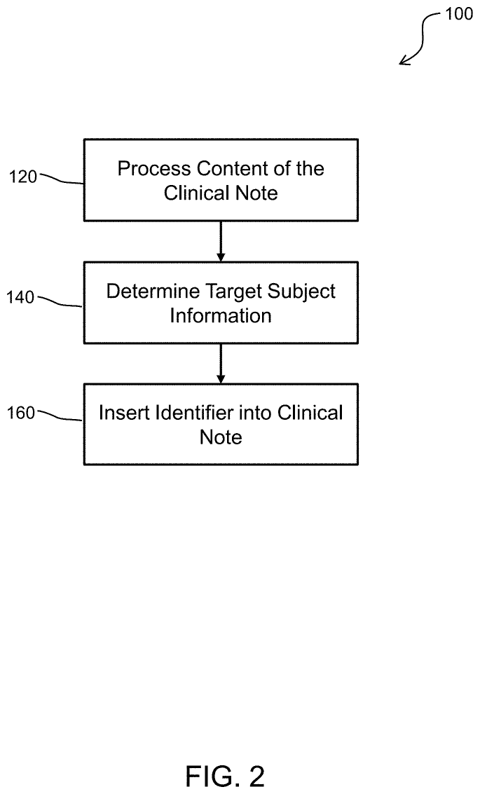

FIG. 2 is a flow diagram of a method 100 for generating an augmented clinical note according to an exemplary embodiment.

At step 120, content of a clinical note corresponding to a subject is processed with a natural language processing algorithm in order to identify a data type. Content of the clinical note may be summarized or textual copied information, produced by a clinician. The natural language processing algorithm identifies a data type relevant to the clinical note. For example, if the clinical note is related to blood pressure of the subject, then the data type may be “blood pressure”.

By utilizing a natural language processing algorithm, a data type of the content of the note clinical may be automatically, quickly and easily determined. In some embodiments, multiple data types may be identified by the natural language processing algorithm when relevant.

It may be advantageous for the natural language processing algorithm to comprise a machine learning algorithm. In this case, the machine learning algorithm is trained using a training algorithm configured to receive an array of training inputs and known outputs. The training inputs comprise training (e.g. historical) clinical notes and the known outputs comprise data types for the training notes. In this way, a data type may be determined which is more relevant to content of the clinical note.

In other words, the machine learning algorithm may be trained using a training algorithm configured to receive an array of training inputs and known outputs for the training inputs. The training inputs may comprise clinical notes (or content of clinical notes) and the known outputs comprise data types that have been previously determined for the clinical notes.

As a result, by inputting content of the clinical note corresponding to the subject into the trained machine learning algorithm, a data type may be obtained.

The machine learning algorithm may comprise an artificial neural network (or, simply, a neural network). The structure of an artificial neural network is inspired by the human brain. Neural networks are comprised of layers, each layer comprising a plurality of neurons. Each neuron comprises a mathematical operation. In particular, each neuron may comprise a different weighted combination of a single type of transformation (e.g. the same type of transformation, sigmoid etc. but with different weightings). In the process of processing input data, the mathematical operation of each neuron is performed on the input data to produce a numerical output, and the outputs of each layer in the neural network are fed into the next layer sequentially. The final layer provides the output.

There are several types of neural network, such as convolutional neural networks (CNNs) and recurrent neural networks (RNNs).

Methods of training a machine-learning algorithm are well known. Typically, such methods comprise obtaining a training dataset, comprising training input data entries and corresponding training output data entries. An initialized machine-learning algorithm is applied to each input data entry to generate predicted output data entries. An error between the predicted output data entries and corresponding training output data entries is used to modify the machine-learning algorithm. This process can be repeated until the error converges, and the predicted output data entries are sufficiently similar (e.g. ±1%) to the training output data entries. This is commonly known as a supervised learning technique.

For example, weightings of the mathematical operation of each neuron may be modified until the error converges. Known methods of modifying a neural network include gradient descent, backpropagation algorithms and so on.

At step 140, target subject information is determined based on the identified data type. In other words, after a data type has been identified, target subject information stored in databases may be selected which corresponds to the identified data type. In this way, information relating to the subject corresponding to the clinical note is determined. By way of example, if the data type is “blood pressure”, the target subject information may be a list of blood pressure readings of the subject. In this way, information which may be useful for reference when reading the clinical note may be identified.

At step 160, an identifier of a location of the target subject information is inserted into the clinical note in order to generate an augmented clinical note. An identifier may be to a location may be inserted, instead of the target subject information itself in order to provide a clinical note that remains concise, while also providing all necessary information. The identifier may then be used by the reader to access (relevant) target subject information.

Accordingly, an augmented clinical note is generated, which contains a reference to (relevant) target subject information. As the identifier is inserted, there may be no need for the creator of the clinical note to duplicate information contained at the location designated by the identifier.

Further, it may be beneficial for the inserted identifier to be configured to be user selectable. In such a case, the method may further comprise communicating the target subject information to a user responsive to the user selecting the identifier. As a result, it may not be necessary for the user to use other systems or platforms to access the target subject information. Indeed, in this way the target subject information may be quickly and easily accessed. By way of example, the identifier may be a hyperlink capable of being selected by the user, and navigating the user to the target subject information.

In some embodiments, communicating the target subject information to the user may include modifying the clinical note to include the target subject information in the clinical note. In other words, communicating the target subject information may embed the information within the clinical note. In alternative embodiments, communicating the target subject information to the user may include producing a pop up, or may navigate the user to a separate page or system. In yet further embodiments, communicating the target subject information to the user may include alternative methods of communication, or may include different methods depending on the format of the target subject information being communicated.

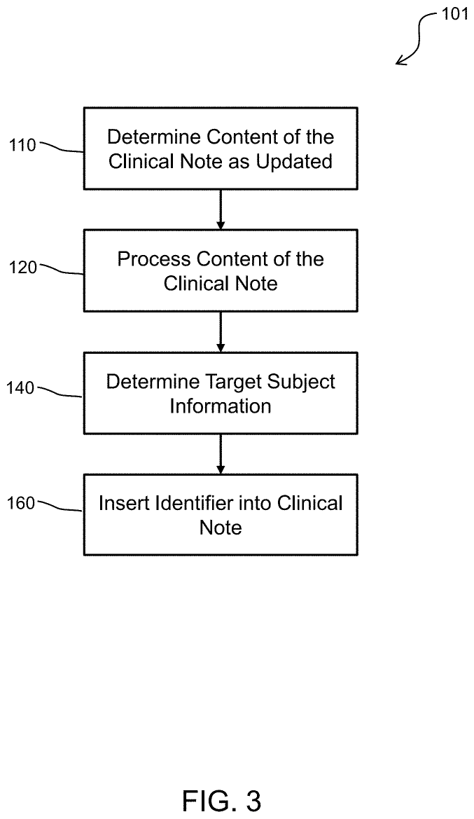

FIG. 3 is a flow diagram of a method 101 for generating an augmented clinical note according to an exemplary embodiment. Steps 120, 140 and 160 are similar to steps 120, 140 and 160 described in relation to FIG. 2, and therefore further explanation is omitted here for the sake of conciseness.

The method further includes a preceding step 110 of determining content of the clinical note as being updated. When the clinical note is determined as updated the steps of processing the content of the clinical note and determining target subject information are performed.

To paraphrase, the clinical note may be continuously monitored, such that it is determined whenever it is amended by addition, deletion or modification. In the case that it is detected that the clinical note has been amended, the data type of the note is identified, as the data type may have changed. Indeed, in this way the target subject information desirable for reference in the clinical note may change.

On the user side, this may be realized as the clinical note having different identifiers inserted as the clinical note is being created (e.g. written or dictate). Accordingly, the author of the clinical note may select an identifier which is appropriate as the clinical note is being written.

By determining when the content of the clinical note has been updated, target subject information may be identified whenever a user adds, subtracts or alters content of the clinical note. As a result, target subject information which is relevant to the current state of the content of the clinical note may be identified. Indeed, by identifying a data type and corresponding target subject information in response to the clinical note being updated, it may be ensured that the identifier to an address of target subject information is appropriate for the given clinical note content.

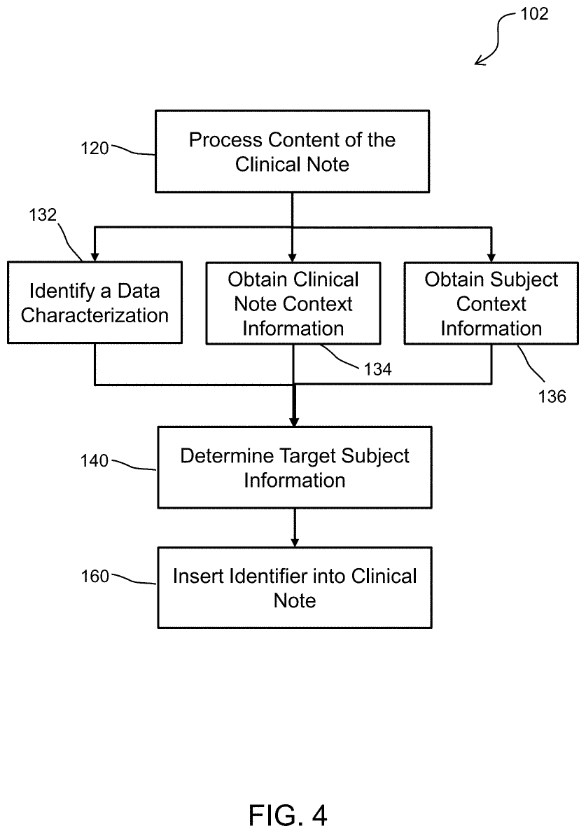

FIG. 4 is a flow diagram of a method 102 for generating an augmented clinical note according to an exemplary embodiment. Steps 120, 140 and 160 are similar to steps 120, 140 and 160 described in relation to FIG. 2, and therefore further explanation is omitted here for the sake of conciseness. The method shown in FIG. 4 further includes steps 132, 134 and 136, but may alternatively include any combination of these steps. Indeed, in some embodiments the method may also include step 110.

In step 132, content of the clinical note is analyzed using the natural language processing algorithm to identify a data characterization. In this case, determining target subject information is further based on the data characterization as well as the data type.

In some cases, the data characterization may indicate the character of the target subject information required. For example, if the clinical note contains the words “latest blood pressure”, then the data characterization may be that the word “latest”. Accordingly, the target subject information may be the latest available blood pressure reading of the subject. As a result, determined target subject information may be more appropriate. Accordingly, the clinical note may be more useful for a reader.

By way of example, the data characterization may comprise one or more of a date stamp, a time stamp, a time window, a variation characteristic, a trend characteristic, a measurement location, a measurement device, a body position of subject, a data storage location, and a measurement characteristic. However, the invention is not limited to these, and it could be any type of data characterization.

In step 134, clinical note context information is obtained. In this case, determining the target subject information is further based on the clinical note context information. Clinical note information may provide useful information for identifying relevant target subject information. Indeed, clinical note context information may be any information related to the writing of the clinical note.

By way of example, the clinical note context information may comprise one or more of a clinical note timestamp, an author, an author role, and a location of where the clinical note was created (e.g. written and/or dictated). However, the invention is not limited to these, and it could be any type of data related to the context of clinical note.

In this way, target subject information may be identified which is more relevant to the content of the clinical note.

In step 136, subject context information is obtained. The subject context information may be obtained from at least one of EMRs, a picture archiving and communication system (PACS), a lab test, and a medical database. In this case, determining the target subject information may be further based on the subject context information. By basing the target subject information on the context of the subject, more relevant target subject information may be identified. For example, if the subject has conditions relating to the heart (a recent cardiac arrest), then it is more likely that the relevant target information will include heart rate readings.

By way of example, the subject context information may comprise one or more of a subject history, a subject measurement, a subject condition, a subject treatment plan, a subject location, a subject alarm setting, a subject alarm history, and a subject early warning score. However, the invention is not limited to these, and it could be any type of data related to the context of subject.

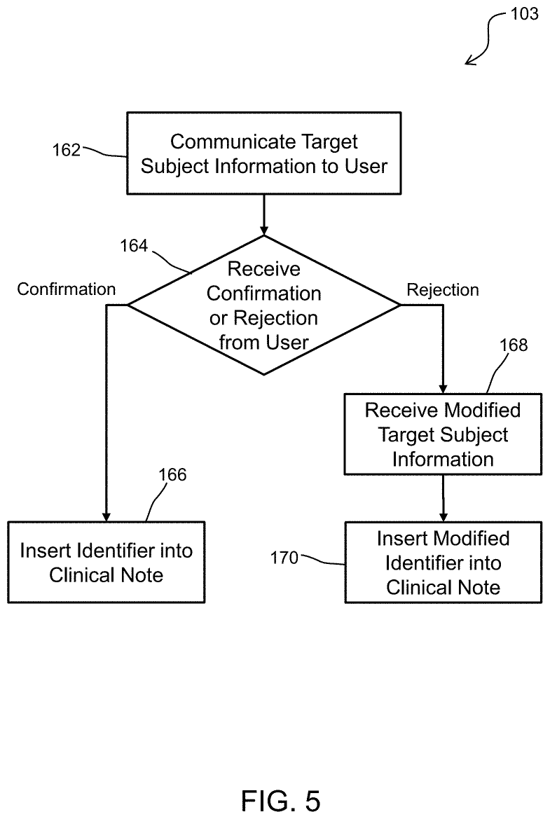

FIG. 5 presents a flow diagram of a method 103 for inserting an identifier into a clinical note according to an aspect of an exemplary embodiment. This method may be used alongside any of the above described methods.

At step 162, the target subject information is communicated to a user. The target subject information may be communicated to the user by a pop up box, or may only be provided to the user upon request. In any case, the user is presented with the target subject information determined by at least a data type of the content of the clinical note.

At step 164, a confirmation or a rejection corresponding to the target subject information from the user is received. In other words, the user decides whether the target subject information would be useful when reading the clinical note. For example, if the clinical note does not require any information, then the user may decide to reject the target subject information. Alternatively, the target subject information may not be relevant to the clinical note and therefore may be rejected.

In some embodiments, a history of confirmations and rejections may be used when determining target subject information.

Responsive to receiving a confirmation from the user, the method moves to step 166. At step 166, the identifier of the location of the target subject information is inserted into the clinical note in order to generate the augmented clinical note.

Responsive to receiving a rejection from the user, the identifier of the location of the target subject information is not inserted into the clinical note. In some embodiments (not shown), this means that no identifier is inserted at all, and the method ends. Indeed, it may be the case that the user expresses that they do not wish to insert an identifier into the clinical note. In some cases it may not be desirable to insert an identifier, and therefore the user may be given the choice.

Alternatively, responsive to receiving a rejection from the user, the method moves onto step 168. At step 168, modified target subject information is received from the user. Indeed, the user may be in a situation where they have certain target subject information in mind, which they want an identifier inserted for. Therefore, the method allows the user to modify the target subject information.

By way of example, should the user wish to insert blood pressure readings for a full day, rather than a single reading as communicated, then it may prove particularly beneficial to give the user the chance to modify the target subject information. Accordingly, target subject information which is more relevant to the clinical note may be determined.

At step 170, an identifier of a location of the modified target subject information is inserted into the clinical note in order to generate the augmented clinical note.

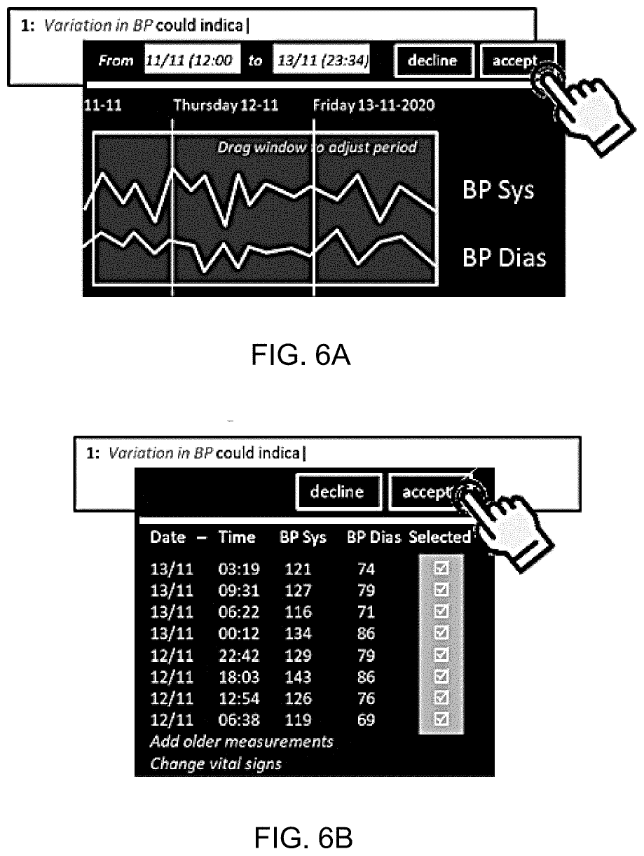

In reference to FIGS. 6A and 6B, there is presented a user selection screen for inserting an identifier into a clinical note.

Specifically, FIG. 6A shows the possibility of an identifier of a location of target subject information including a graph of subject blood pressure readings. The user may choose to accept, decline, or modify the target subject information.

Likewise, FIG. 6B shows the possibility of an identifier of a location of target subject information including a table of subject blood pressure readings. The user may choose to accept, decline, or modify the target subject information

By way of explanation, while typing the clinical note, embodiments of the invention may analyse the content of the note to automatically propose identifiers to target subject information. As the content of the clinical note is added to, the suggested target subject information may be continuously updated based on the added content in the clincal note.

The user may very easily accept or decline the proposed identifier. The user may also quickly and easily adjust the exact content of the target subject information, for example by selecting another vital sign measurement or time period.

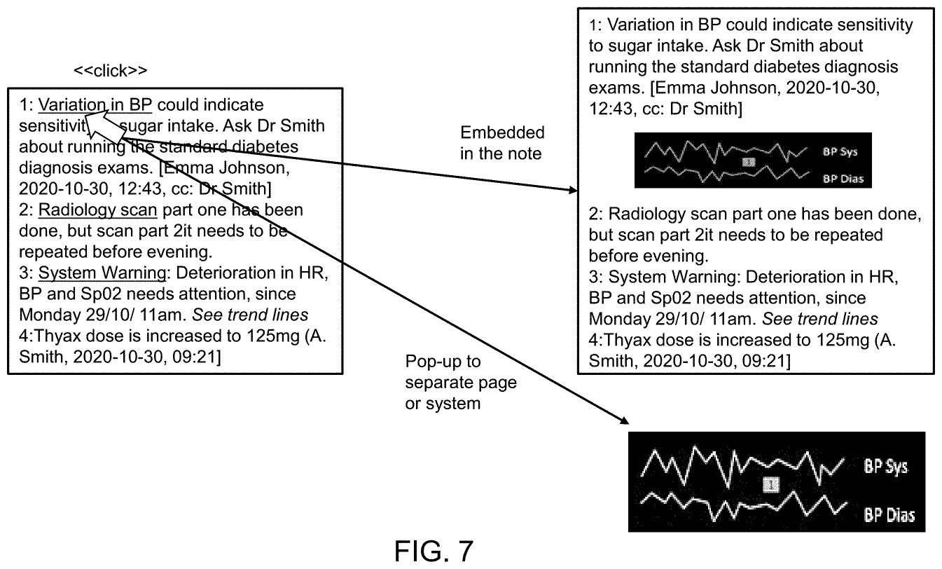

FIG. 7 depicts uses of an identifier inserted into a clinical note. In particular, when reading the clinical note the user may select the identifier (in this case a hyperlink) to navigate to the target subject information. In some embodiments, depending on the type of target subject information or preference of the user, this information can be either shown embedded in the note or in a separate window or system.

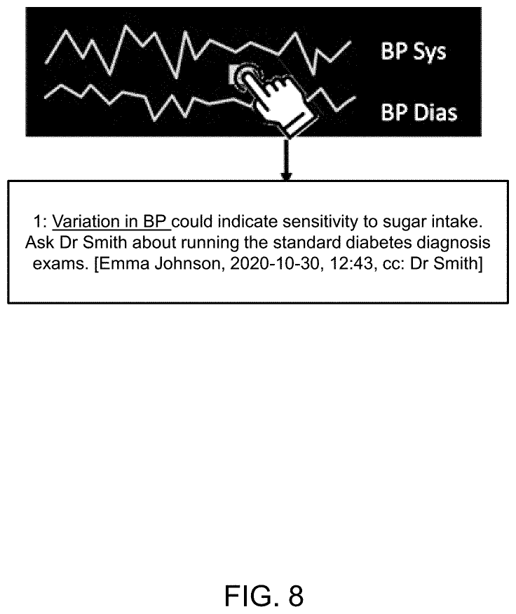

FIG. 8 depicts a use of an identifier inserted into subject information. Specifically, the target subject information itself, in the location referenced by the identifier, may show an indication that it is referenced in a clinical note. The indication may also include an identifier to a location of the clinical note.

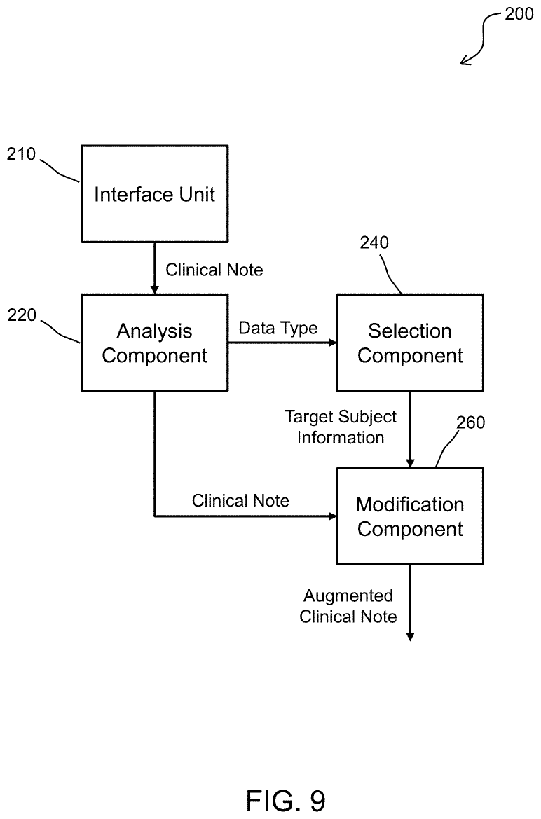

Turning to FIG. 9, there is presented a block diagram of a system 200 for generating an augmented clinical note according to an exemplary embodiment. The system comprises an analysis component 220, a selection component 240, and a modification component 260. Optionally, the system also includes an interface 210.

The analysis component 220 is configured to process, with a natural language processing algorithm, content of a clinical note corresponding to a subject in order to identify a data type. The selection component 240 is configured to determine target subject information based on the identified data type. The modification component 260 is configured to insert an identifier of a location of the target subject information into the clinical note in order to generate an augmented clinical note.

In this way, the system 200 may provide a means to generate augmented clinical notes by inserting an identifier to (relevant) target subject information. Accordingly, the speed of creating a clinical note may be improved by reducing the rate of errors. Also, the speed of reading a clinical note may be improved as referenced information is easily available, and the rate of errors in referencing may be suppressed.

Further, in some embodiments there may be provided an interface unit 210 configured to determine content of the clinical note as being updated. In the case that the interface unit 210 is provided, the analysis 220 component may be further configured to analyze the content of the clinical note, and the selection component 240 may be further configured to determine target subject information, responsive to the interface unit determining that the content has been updated.

Accordingly, the system 200 may continuously monitor the clinical note and provide updated target subject information when the content of the note is altered. This means that more relevant target subject information may be provided at any given time, even when the clinical note is being written.

A single processor or other unit may fulfil the functions of several items recited in the claims.

A computer program may be stored/distributed on a suitable medium, such as an optical storage medium or a solid-state medium supplied together with or as part of other hardware, but may also be distributed in other forms, such as via the Internet or other wired or wireless telecommunication systems.

The mere fact that certain measures are recited in mutually different dependent claims does not indicate that a combination of these measures cannot be used to advantage.

Any reference signs in the claims should not be construed as limiting the scope.

1. A method for generating an augmented clinical note, the method comprising:
processing, with a natural language processing algorithm, content of a clinical note corresponding to a subject in order to identify a data type;
determining target subject information based on the identified data type; and
inserting an identifier of a location of the target subject information into the clinical note in order to generate an augmented clinical note.

2. The method of claim 1, further comprising a preceding step of determining content of the clinical note as being updated, and wherein the steps of processing the content of the clinical note and determining target subject information are performed responsive to determining that the content has been updated.

3. The method of claim 1, wherein the natural language processing algorithm comprises a machine learning algorithm that is trained using a training algorithm configured to receive an array of training inputs and known outputs, wherein the training inputs comprise clinical notes and the known outputs comprise data types.

4. The method of claim 1, wherein the inserted identifier is configured to be user selectable, and wherein the method further comprises communicating the target subject information to a user responsive to the user selecting the identifier.

5. The method of claim 1, wherein the method further comprises analyzing content of the clinical note using the natural language processing algorithm to identify a data characterization, and wherein determining target subject information is further based on the data characterization.

6. The method of claim 5, wherein the data characterization comprises one or more of a date stamp, a time stamp, a time window, a variation characteristic, a trend characteristic, a measurement location, a measurement device, a body position of subject, a data storage location, and a measurement characteristic.

7. The method of claim 1, wherein determining the target subject information is further based on subject context information, the subject context information comprising one or more of a subject history, a subject measurement, a subject condition, a subject treatment plan, a subject location, a subject alarm setting, a subject alarm history, and a subject early warning score.

8. The method of claim 7, wherein the method further comprises obtaining subject context information from at least one of:
an electronic medical record, EMR;
a picture archiving and communication system, PACS;
a lab test; and
a medical database.

9. The method of claim 1, wherein determining the target subject information is further based on clinical note context information.

10. The method of claim 9, wherein the clinical note context information comprises one or more of a clinical note timestamp, an author, an author role, and a location of where the clinical note was created.

11. The method of claim 1, wherein inserting the identifier to the location of the target subject information comprises:
communicating the target subject information to a user;
receiving a confirmation or a rejection corresponding to the target subject information from the user; and
inserting the identifier of the location of the target subject information into the clinical note in order to generate the augmented clinical note, responsive to receiving confirmation from the user.

12. The method of claim 11, further comprising:
receiving modified target subject information from the user responsive to communicating the relevant subject information to the user; and
inserting an identifier of a location of the modified target subject information into the clinical note in order to generate the augmented clinical note.

13. A computer program comprising computer program code means which is adapted, when said computer program is run on a computer, to implement the method of claim 1.

14. A system for generating an augmented clinical note, the system comprising:
an analysis component configured to process, with a natural language processing algorithm, content of a clinical note corresponding to a subject in order to identify a data type;
a selection component configured to determine target subject information based on the identified data type; and
a modification component configured to insert an identifier of a location of the target subject information into the clinical note in order to generate an augmented clinical note.

15. The system of claim 14, further comprising an interface unit configured to determine content of the clinical note as being updated, and
wherein the analysis component is further configured to analyze the content of the clinical note, and the selection component is further configured to determine target subject information, responsive to the interface unit determining that the content has been updated.
